(19) United States
(12) Patent Application Publication
(10) Pub. No.: US 2025/0045976 A1
(43) Pub. Date: Feb. 6, 2025

(54) Rendering User Emotion in a Metaverse for User Awareness

(71) Applicant: International Business Machines Corporation, Armonk, NY (US)

(72) Inventors: Jeremy R. Fox, Georgetown, TX (US); Alexander Reznicek, Troy, NY (US); Bahman Hekmatshoartabari, White Plains, NY (US); Martin G. Keen, Cary, NC (US)

(21) Appl. No.: 18/362,489

(22) Filed: Jul. 31, 2023

(51) Int. Cl. G06T 11/00; A61B 5/00; A61B 5/16; G06F 3/01; G06N 3/0475; G06N 3/08; G10L 25/63

(52) U.S. Cl. CPC G06T 11/00; A61B 5/165; A61B 5/4266; A61B 5/6803; G06F 3/011; G06N 3/0475; G06N 3/08; G10L 25/63; A61B 5/01; A61B 5/4803; A61B 2562/063

(57) ABSTRACT

Rendering an emotional state of a virtual reality headset user is provided. An emotion feature vector predicting a current emotional state of a user of a virtual reality headset is mapped to a matching set of existing avatar vectors a mapping function. A best matching avatar vector is selected from the matching set of existing avatar vectors based on determining that values of the best matching avatar vector most closely match values of the emotion feature vector predicting the current emotional state of the user of the virtual reality headset. An avatar associated with the user is rendered in a metaverse consistent with the current emotional state of the user of the virtual reality headset based on the best matching avatar vector to the emotion feature vector predicting the current emotional state of the user.

### BACKGROUND

The disclosure relates generally to virtual environments and more specifically to rendering user emotion in a metaverse for user awareness.

Metaverse is a broad term that generally refers to shared three-dimensional virtual world environments, which people can access via the internet or some other type of network. The term metaverse can refer to digital spaces which are made lifelike by use of virtual reality headset devices. Virtual reality is a digital environment that shuts out the real world. The field of virtual reality is rapidly growing and being applied in a wide range of areas, such as, for example, remote working, meeting, training, entertaining, marketing, real estate, and the like. Some people also use the term metaverse to describe gaming worlds, in which users have an avatar that can walk around and interact with other avatars. An avatar is a graphical representation of a user or the user's character or persona. Avatars can be two-dimensional icons or can take the form of a three-dimensional model as used in online worlds and video games. Like the internet, the metaverse helps people connect when they are not physically located in the same place and can provide a feeling of togetherness. Metaverse development is often linked to advancing virtual reality technology due to the increasing demand for immersion.

### SUMMARY

According to one illustrative embodiment, a computer-implemented method for rendering an emotional state of a virtual reality headset user is provided. An emotion feature vector predicting a current emotional state of a user of a virtual reality headset is mapped to a matching set of existing avatar vectors a mapping function. A best matching avatar vector is selected from the matching set of existing avatar vectors based on determining that values of the best matching avatar vector most closely match values of the emotion feature vector predicting the current emotional state of the user of the virtual reality headset. An avatar associated with the user is rendered in a metaverse consistent with the current emotional state of the user of the virtual reality headset based on the best matching avatar vector to the emotion feature vector predicting the current emotional state of the user. According to other illustrative embodiments, a computing environment and computer program product for rendering an emotional state of a virtual reality headset user are provided.

### BRIEF DESCRIPTION OF THE DRAWINGS

FIG. 1 is a pictorial representation of a computing environment in which illustrative embodiments may be implemented;

FIG. 2 is a diagram illustrating an example of a user emotion prediction process in accordance with an illustrative embodiment;

FIGS. 3A-3B are a diagram illustrating an example of an alternative user emotion prediction process in accordance with an illustrative embodiment;

FIG. 4 is a diagram illustrating an example of a rendering component in accordance with an illustrative embodiment;

FIG. 5 is a diagram illustrating an example of a virtual reality headset in accordance with an illustrative embodiment;

FIG. 6 is a diagram illustrating an example of a biosensor in accordance with an illustrative embodiment;

FIG. 7 is a diagram illustrating an example of an alternative biosensor in accordance with an illustrative embodiment;

FIG. 8 is a diagram illustrating an example of another alternative biosensor in accordance with an illustrative embodiment;

FIG. 9 is a diagram illustrating an example of another alternative biosensor in accordance with an illustrative embodiment;

FIG. 10 is a diagram illustrating an example of another alternative biosensor in accordance with an illustrative embodiment; and

FIGS. 11A-11B are a flowchart illustrating a process for rendering an emotional state of a virtual reality headset user in a virtual reality environment in accordance with an illustrative embodiment.

### DETAILED DESCRIPTION

Various aspects of the present disclosure are described by narrative text, flowcharts, block diagrams of computer systems and/or block diagrams of the machine logic included in computer program product (CPP) embodiments. With respect to any flowcharts, depending upon the technology involved, the operations can be performed in a different order than what is shown in a given flowchart. For example, again depending upon the technology involved, two operations shown in successive flowchart blocks may be performed in reverse order, as a single integrated step, concurrently, or in a manner at least partially overlapping in time.

A computer program product embodiment (“CPP embodiment” or “CPP”) is a term used in the present disclosure to describe any set of one, or more, storage media (also called “mediums”) collectively included in a set of one, or more, storage devices that collectively include machine readable code corresponding to instructions and/or data for performing computer operations specified in a given CPP claim. A “storage device” is any tangible device that can retain and store instructions for use by a computer processor. Without limitation, the computer readable storage medium may be an electronic storage medium, a magnetic storage medium, an optical storage medium, an electromagnetic storage medium, a semiconductor storage medium, a mechanical storage medium, or any suitable combination of the foregoing. Some known types of storage devices that include these mediums include: diskette, hard disk, random access memory (RAM), read-only memory (ROM), erasable programmable read-only memory (EPROM or Flash memory), static random access memory (SRAM), compact disc read-only memory (CD-ROM), digital versatile disk (DVD), memory stick, floppy disk, mechanically encoded device (such as punch cards or pits/lands formed in a major surface of a disc), or any suitable combination of the foregoing. A computer readable storage medium, as that term is used in the present disclosure, is not to be construed as storage in the form of transitory signals per se, such as radio waves or other freely propagating electromagnetic waves, electromagnetic waves propagating through a waveguide, light pulses passing through a fiber optic cable, electrical signals communicated through a wire, and/or other transmission media. As will be understood by those of skill in the art, data is typically moved at some occasional points in time during normal operations of a storage device, such as during access, de-fragmentation or garbage collection, but this does not render the storage device as transitory because the data is not transitory while it is stored.

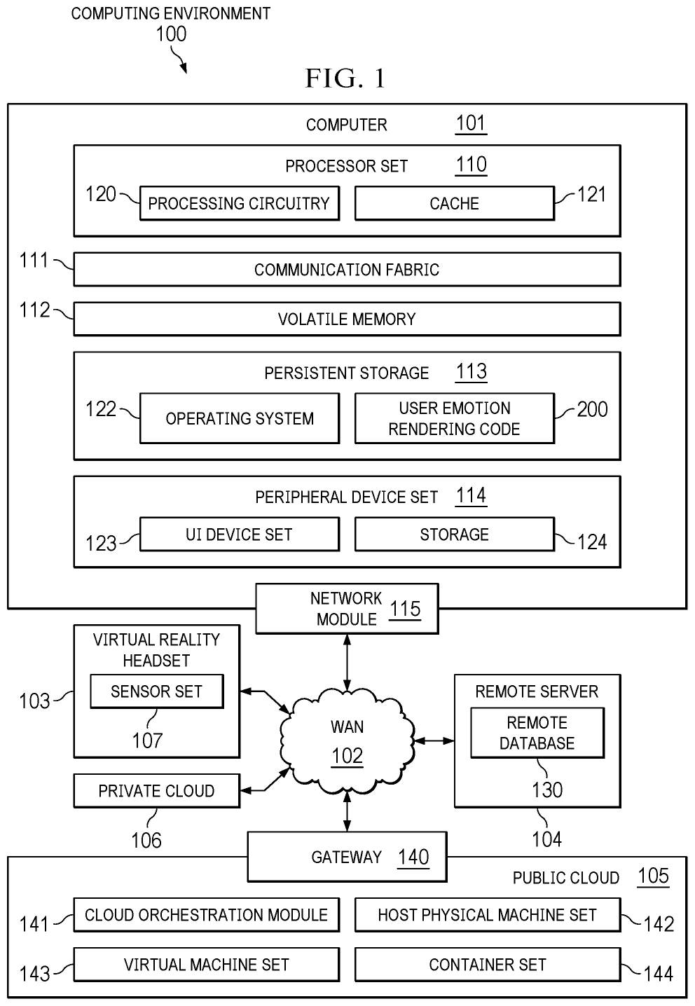

With reference now to the figures, and in particular, with reference to FIGS. 1-10, diagrams of data processing environments are provided in which illustrative embodiments may be implemented. It should be appreciated that FIGS. 1-10 are only meant as examples and are not intended to assert or imply any limitation with regard to the environments in which different embodiments may be implemented. Many modifications to the depicted environments may be made.

FIG. 1 shows a pictorial representation of a computing environment in which illustrative embodiments may be implemented. Computing environment 100 contains an example of an environment for the execution of at least some of the computer code involved in performing the inventive methods of illustrative embodiments, such as user emotion rendering code 200.

For example, user emotion rendering code 200 detects and extracts data via a monolithically integrated array of biosensors, which target biomarkers in sweat for emotion detection. The monolithically integrated array of biosensors is attached to a virtual reality device (e.g., virtual reality headset) of a user. User emotion rendering code 200 utilizes a combination of outputs from a multi-biomarker biosensor array, a body temperature sensor, and a speech emotion sensor to determine the emotional state or mood of the user. Thus, user emotion rendering code 200 provides consistent and accurate user emotion detection as user emotion rendering code 200 utilizes one or more constant biomarker contact points on the skin of the user (e.g., forehead, base of neck, bridge of nose, or the like) where stress biomarkers can be detected leading to more accurate avatar representation of the user in the metaverse (e.g., virtual world environment) by user emotion rendering code 200. User emotion rendering code 200 utilizes machine learning models, such as, for example, discriminative neural networks and generative neural networks, for the mapping to and the rendering of avatars associated with users in the metaverse based on determined emotional states of the users.

In addition to user emotion rendering code 200, computing environment 100 includes, for example, computer 101, wide area network (WAN) 102, virtual reality headset 103, remote server 104, public cloud 105, and private cloud 106. In this embodiment, computer 101 includes processor set 110 (including processing circuitry 120 and cache 121), communication fabric 111, volatile memory 112, persistent storage 113 (including operating system 122 and user emotion rendering code 200, as identified above), peripheral device set 114 (including user interface (UI) device set 123 and storage 124), and network module 115. Remote server 104 includes remote database 130. Public cloud 105 includes gateway 140, cloud orchestration module 141, host physical machine set 142, virtual machine set 143, and container set 144.

Computer 101 may take the form of a mainframe computer, quantum computer, desktop computer, laptop computer, tablet computer, or any other form of computer now known or to be developed in the future that is capable of, for example, running a program, accessing a network, and querying a database, such as remote database 130. As is well understood in the art of computer technology, and depending upon the technology, performance of a computer-implemented method may be distributed among multiple computers and/or between multiple locations. On the other hand, in this presentation of computing environment 100, detailed discussion is focused on a single computer, specifically computer 101, to keep the presentation as simple as possible. Computer 101 may be located in a cloud, even though it is not shown in a cloud in FIG. 1. On the other hand, computer 101 is not required to be in a cloud except to any extent as may be affirmatively indicated.

Processor set 110 includes one, or more, computer processors of any type now known or to be developed in the future. Processing circuitry 120 may be distributed over multiple packages, for example, multiple, coordinated integrated circuit chips. Processing circuitry 120 may implement multiple processor threads and/or multiple processor cores. Cache 121 is memory that is located in the processor chip package(s) and is typically used for data or code that should be available for rapid access by the threads or cores running on processor set 110. Cache memories are typically organized into multiple levels depending upon relative proximity to the processing circuitry. Alternatively, some, or all, of the cache for the processor set may be located “off chip.” In some computing environments, processor set 110 may be designed for working with qubits and performing quantum computing.

Computer readable program instructions are typically loaded onto computer 101 to cause a series of operational steps to be performed by processor set 110 of computer 101 and thereby effect a computer-implemented method, such that the instructions thus executed will instantiate the methods specified in flowcharts and/or narrative descriptions of computer-implemented methods included in this document (collectively referred to as “the inventive methods”). These computer readable program instructions are stored in various types of computer readable storage media, such as cache 121 and the other storage media discussed below. The program instructions, and associated data, are accessed by processor set 110 to control and direct performance of the inventive methods. In computing environment 100, at least some of the instructions for performing the inventive methods of illustrative embodiments may be stored in user emotion rendering code 200 in persistent storage 113.

Communication fabric 111 is the signal conduction path that allows the various components of computer 101 to communicate with each other. Typically, this fabric is made of switches and electrically conductive paths, such as the switches and electrically conductive paths that make up buses, bridges, physical input/output ports, and the like. Other types of signal communication paths may be used, such as fiber optic communication paths and/or wireless communication paths.

Volatile memory 112 is any type of volatile memory now known or to be developed in the future. Examples include dynamic type random access memory (RAM) or static type RAM. Typically, volatile memory 112 is characterized by random access, but this is not required unless affirmatively indicated. In computer 101, the volatile memory 112 is located in a single package and is internal to computer 101, but, alternatively or additionally, the volatile memory may be distributed over multiple packages and/or located externally with respect to computer 101.

Persistent storage 113 is any form of non-volatile storage for computers that is now known or to be developed in the future. The non-volatility of this storage means that the stored data is maintained regardless of whether power is being supplied to computer 101 and/or directly to persistent storage 113. Persistent storage 113 may be a read only memory (ROM), but typically at least a portion of the persistent storage allows writing of data, deletion of data, and re-writing of data. Some familiar forms of persistent storage include magnetic disks and solid-state storage devices. Operating system 122 may take several forms, such as various known proprietary operating systems or open-source Portable Operating System Interface-type operating systems that employ a kernel. User emotion rendering code 200 includes at least some of the computer code involved in performing the inventive methods of illustrative embodiments.

Peripheral device set 114 includes the set of peripheral devices of computer 101. Data communication connections between the peripheral devices and the other components of computer 101 may be implemented in various ways, such as Bluetooth connections, Near-Field Communication (NFC) connections, connections made by cables (such as universal serial bus (USB) type cables), insertion-type connections (for example, secure digital (SD) card), connections made through local area communication networks, and even connections made through wide area networks such as the internet. In various embodiments, UI device set 123 may include components such as a display screen, speaker, microphone, wearable devices (such as smart glasses and smart watches), keyboard, mouse, touchpad, and haptic devices. Storage 124 is external storage, such as an external hard drive, or insertable storage, such as an SD card. Storage 124 may be persistent and/or volatile. In some embodiments, storage 124 may take the form of a quantum computing storage device for storing data in the form of qubits. In embodiments where computer 101 is required to have a large amount of storage (for example, where computer 101 locally stores and manages a large database) then this storage may be provided by peripheral storage devices designed for storing very large amounts of data, such as a storage area network (SAN) that is shared by multiple, geographically distributed computers.

Network module 115 is the collection of computer software, hardware, and firmware that allows computer 101 to communicate with other computers through WAN 102. Network module 115 may include hardware, such as modems or Wi-Fi signal transceivers, software for packetizing and/or de-packetizing data for communication network transmission, and/or web browser software for communicating data over the internet. In some embodiments, network control functions and network forwarding functions of network module 115 are performed on the same physical hardware device. In other embodiments (for example, embodiments that utilize software-defined networking (SDN)), the control functions and the forwarding functions of network module 115 are performed on physically separate devices, such that the control functions manage several different network hardware devices. Computer readable program instructions for performing the inventive methods can typically be downloaded to computer 101 from an external computer or external storage device through a network adapter card or network interface included in network module 115.

WAN 102 is any wide area network (for example, the internet) capable of communicating computer data over non-local distances by any technology for communicating computer data, now known or to be developed in the future. In some embodiments, the WAN 102 may be replaced and/or supplemented by local area networks (LANs) designed to communicate data between devices located in a local area, such as a Wi-Fi network. The WAN and/or LANs typically include computer hardware such as copper transmission cables, optical transmission fibers, wireless transmission, routers, firewalls, switches, gateway computers, and edge servers.

Virtual reality headset 103 is a data processing device that is used and controlled by an end user (e.g., a user of the metaverse services provided by computer 101), and can include similar components as those discussed above in connection with computer 101. Virtual reality headset 103 typically receives useful data from the operations of computer 101. For example, in a hypothetical case where computer 101 is designed to provide a metaverse (e.g., virtual reality environment) to the end user, this metaverse would typically be provided via network module 115 of computer 101 through WAN 102 to virtual reality headset 103. In this way, virtual reality headset 103 can display, or otherwise present, the metaverse to the end user. Virtual reality headset 103 includes sensor set 107. Sensor set 107 is made up of a group of sensors that is used by at least one of virtual reality headset 103 and computer 101. For example, one sensor can be a multi-biomarker biosensor array, another sensor can be a speech emotion sensor, and yet another sensor can be a body temperature sensor for detecting a current emotional state of a user of virtual reality headset 103. Furthermore, virtual reality headset 103 can include user emotion rendering code 200 in addition to, or instead of, computer 101. Moreover, virtual reality headset 103 can represent a plurality of different virtual reality headsets in computing environment 100.

Remote server 104 is any computer system that serves at least some data and/or functionality to computer 101. Remote server 104 may be controlled and used by the same entity that operates computer 101. Remote server 104 represents the machine(s) that collect and store helpful and useful data for use by other computers, such as computer 101. For example, in a hypothetical case where computer 101 is designed and programmed to provide a metaverse based on historical data, then this historical data may be provided to computer 101 from remote database 130 of remote server 104.

Public cloud 105 is any computer system available for use by multiple entities that provides on-demand availability of computer system resources and/or other computer capabilities, especially data storage (cloud storage) and computing power, without direct active management by the user. Cloud computing typically leverages sharing of resources to achieve coherence and economics of scale. The direct and active management of the computing resources of public cloud 105 is performed by the computer hardware and/or software of cloud orchestration module 141. The computing resources provided by public cloud 105 are typically implemented by virtual computing environments that run on various computers making up the computers of host physical machine set 142, which is the universe of physical computers in and/or available to public cloud 105. The virtual computing environments (VCEs) typically take the form of virtual machines from virtual machine set 143 and/or containers from container set 144. It is understood that these VCEs may be stored as images and may be transferred among and between the various physical machine hosts, either as images or after instantiation of the VCE. Cloud orchestration module 141 manages the transfer and storage of images, deploys new instantiations of VCEs and manages active instantiations of VCE deployments. Gateway 140 is the collection of computer software, hardware, and firmware that allows public cloud 105 to communicate through WAN 102.

Some further explanation of virtualized computing environments (VCEs) will now be provided. VCEs can be stored as “images.” A new active instance of the VCE can be instantiated from the image. Two familiar types of VCEs are virtual machines and containers. A container is a VCE that uses operating-system-level virtualization. This refers to an operating system feature in which the kernel allows the existence of multiple isolated user-space instances, called containers. These isolated user-space instances typically behave as real computers from the point of view of programs running in them. A computer program running on an ordinary operating system can utilize all resources of that computer, such as connected devices, files and folders, network shares, CPU power, and quantifiable hardware capabilities. However, programs running inside a container can only use the contents of the container and devices assigned to the container, a feature which is known as containerization.

Private cloud 106 is similar to public cloud 105, except that the computing resources are only available for use by a single entity. While private cloud 106 is depicted as being in communication with WAN 102, in other embodiments a private cloud may be disconnected from the internet entirely and only accessible through a local/private network. A hybrid cloud is a composition of multiple clouds of different types (for example, private, community or public cloud types), often respectively implemented by different vendors. Each of the multiple clouds remains a separate and discrete entity, but the larger hybrid cloud architecture is bound together by standardized or proprietary technology that enables orchestration, management, and/or data/application portability between the multiple constituent clouds. In this embodiment, public cloud 105 and private cloud 106 are both part of a larger hybrid cloud.

As used herein, when used with reference to items, “a set of” means one or more of the items. For example, a set of clouds is one or more different types of cloud environments. Similarly, “a number of,” when used with reference to items, means one or more of the items. Moreover, “a group of” or “a plurality of” when used with reference to items, means two or more of the items.

Further, the term “at least one of,” when used with a list of items, means different combinations of one or more of the listed items may be used, and only one of each item in the list may be needed. In other words, “at least one of” means any combination of items and number of items may be used from the list, but not all of the items in the list are required. The item may be a particular object, a thing, or a category.

For example, without limitation, “at least one of item A, item B, or item C” may include item A, item A and item B, or item B. This example may also include item A, item B, and item C or item B and item C. Of course, any combinations of these items may be present. In some illustrative examples, “at least one of” may be, for example, without limitation, two of item A; one of item B; and ten of item C; four of item B and seven of item C; or other suitable combinations.

There are times in real life when a person's true emotional state, mood, or feelings are misjudged by others. For example, a person arguing passionately about a particular topic may appear angry to others, but in reality, the user is not angry at all. The typical advice in real life is for the person to “look in the mirror” to see if the person's face or body posture is portraying anger to the others. However, this does not work in the metaverse where there is no “mirror” for self-examination, and interaction is not limited to just a small circle of people.

Illustrative embodiments render an avatar corresponding to a user that matches a current emotional state of that particular user in response to illustrative embodiments determining the emotional state of that particular user based on a combination of modalities. The combination of modalities includes, for example, a multi-biomarker biosensor array, a body temperature sensor, a speech emotion sensor, and the like. The multi-biomarker biosensor array can detect biomarkers, such as, for example, cortisol, dopamine, neuropeptide Y, and the like in sweat of a user. Illustrative embodiments utilize the multi-biomarker biosensor array in conjunction with the other modalities (e.g., body temperature sensor and speech emotion sensor) to increase the overall predictive accuracy of classifying the current emotional state of the user. Illustrative embodiments also utilize the body temperature sensor to help detect a range of user emotions. In addition, illustrative embodiments utilize the speech emotion sensor to detect emotion in the user's speech. Even though each of these modalities individually may not provide an accurate assessment of user emotion, illustrative embodiments combine the outputs of these modalities to increase the overall predictive classification accuracy. Thus, illustrative embodiments combine the outputs of these modalities (e.g., multi-biomarker biosensor array, body temperature sensor, and speech emotion sensor) to characterize the emotional state of the user in the metaverse for the user to understand the emotion being projected by the user to other users in the metaverse.

It should be noted that the measurement of stress biomarkers in sweat (e.g., cortisol, dopamine, neuropeptide Y, and the like) has been widely studied. In addition, the identification and measurement of biomarkers for detecting basic emotions, such as, for example, fear, disgust, joy, anger, sadness, and surprise, has also been widely studied. For example, the body releases cortisol, which is a steroid hormone, in response to stress. Cortisol increases blood sugar, increases blood pressure, and prepares the body for the fight-or-flight response by increasing heart rate and blood flow to the muscles. The body also releases dopamine, which is known as the “feel good” neurotransmitter and is part of the body's reward system, to reward a person when doing activities needed to survive, such as the fight-or-flight response during fear or stress, eating, exercising, and the like. The body (i.e., the hypothalamus) also releases neuropeptide Y in response to stress. Neuropeptide Y increases heart rate, blood pressure, and food intake, and stimulates the release of cortisol.

Currently, no solution exists that can reliably classify emotions based on biomarkers alone. Generally, this is because of two issues. The first issue is in order for a biosensor device, such as, for example, an ion-sensitive transistor, to selectively respond to a target biomarker (i.e., detect the presence and concentration of a target biomarker without responding to non-target biomarkers), the sensing surface of the biosensor device needs to be functionalized with the proper chemistry. However, in practice, a sensing surface functionalized for a target biomarker produces a measurable response to non-target biomarkers as well. The second issue is that a biomarker may correlate to more than one emotion, and more than one biomarker may correlate to the same emotion. No well-established rules exist that can exhaustively correlate emotions with biomarkers. This is not because of the lack of correlations, but rather because of the lack of sufficient scientific study to establish the correlation rules.

Illustrative embodiments take into account and address the two issues above. For example, illustrative embodiments utilize an array of densely packed cross-reactive biosensor devices where the sensing surfaces of respective biosensor devices are functionalized differently, enabling the biosensor devices to produce correlated responses to different biomarkers. Illustrative embodiments input the biosensor outputs into a corresponding machine learning model, such as, for example, a neural network, to classify the current emotions of the user based on the collective or aggregate output of the biosensor array rather than individual outputs of differently functionalized biosensors. The corresponding neural network learns correlations (i.e., biosensor output patterns) by observing the data only, without needing any correlation rules during training. As a result, non-ideal biosensor selectivity is not an issue for illustrative embodiments because the neural network classification of user emotions is based on the collective biosensor output patterns rather than the individual biosensor outputs. In other words, illustrative embodiments can utilize any type of biomarker biosensor.

Moreover, illustrative embodiments also detect body temperature and speech emotion as additional modalities to increase the overall emotion classification accuracy of illustrative embodiments. For example, one illustrative embodiment utilizes one of a straight average or a weighted average of the different modalities (i.e., biosensor array output, body temperature sensor output, and speech emotion sensor output) to classify the user emotion. As another example, an alternative illustrative embodiments utilizes a manager neural network to perform end-to-end classification of the emotional state of the user.

The values of an emotion feature vector (e.g., joy, fear, . . . stress) represent the degrees of confidence for the accurate classification of the user's current emotional state. For example, a certain body temperature output in a certain situation may generate an emotion classification confidence of (0.3, 0.5, . . . 0.5), which means that the neural network (e.g., a long short term memory neural network) has 30% confidence that the emotion is (or includes) joy, 50% fear, and 50% stress. This emotion classification confidence, which is based on body temperature alone, does not mean much by itself other than that fear and stress are probably more likely than joy. However, if the biosensor array output in the same situation generates an emotion classification confidence of (0.6, 0.6, . . . 0.3), which means that joy at a 60% confidence level and fear at a 60% confidence level are more likely than stress at a 30% confidence level, then illustrative embodiments can conclude that fear is probably the most likely, or dominant, current user emotion.

Further, speech emotion can contribute to the overall emotion classification as well. For example, one illustrative embodiment can place a higher weight on biosensor array emotion classification and a lesser weight on body temperature emotion classification for all or certain features (e.g., illustrative embodiments assign a high weight for stress classification by the biosensor array because biosensors are known to be good at detecting cortisone). An alternative illustrative embodiment utilizes the manager neural network to learn and assign different weights to the different outputs of the different modalities (i.e., multi-biomarker biosensor array, body temperature sensor, and speech emotion sensor) during training of the manager neural network.

Illustrative embodiments input each of the sensor outputs into a corresponding neural network for user emotion classification. Inputs into a particular neural network are quantities sampled from sensor values (i.e., measured features such as voltages or readout currents) measured and then digitized by analog-to-digital converters, or features extracted from the measurements using signal processing techniques. The extracted features may or may not have physical meanings. Examples of features with physical meanings include, for example, output impedance, self-gain, subthreshold slope, and the like.

In one illustrative embodiment, an inference engine implemented as an application-specific integrated circuit, which is either fabricated monolithically with a sensor chip or fabricated separately and integrated heterogeneously (e.g., bonded to the sensor chip), performs the neural network computations. Prior to operation, illustrative embodiments train the multi-biomarker biosensor array by measuring the output of the multi-biomarker biosensor array in response to various biomarkers and adjusting the neural network parameters to minimize the error in the results of the user emotion classification based on known training techniques, such as, for example, back-propagation.

Illustrative embodiments utilize a plurality of neural networks to process biomarker data (e.g., biomarker types and concentrations), tokenized speech data, and body temperature data collected from the plurality of sensors attached to the virtual reality headset. For example, illustrative embodiment can utilize a recurrent neural network to process a time series of biomarker data output from the multi-biomarker biosensor array. Illustrative embodiments train the recurrent neural network to recognize patterns in the biomarker data that correspond to different user emotional states. In addition, illustrative embodiments can utilize a convolutional neural network to process the tokenized speech data output from the speech emotion sensor. Illustrative embodiments train the convolutional neural network to recognize patterns in the tokenized speech data that correspond to different user emotional states. Further, illustrative embodiments can utilize a long short term memory neural network to process the temperature data output by the body temperature sensor. Illustrative embodiments train the long short term memory neural network to recognize patterns in the temperature data that correspond to different user emotional states. Illustrative embodiments may utilize, for example, a decision tree to preprocess the temperature data. Illustrative embodiments train the decision tree to recognize patterns in the temperature data that correspond to different user emotional states.

It should be noted that illustrative embodiments pretrain each respective neural network in isolation so that illustrative embodiments utilize the biomarker data to train the recurrent neural network, the tokenized speech data to train the convolutional neural network, and the temperature data to train the long short term memory neural network. Furthermore, illustrative embodiments can perform the pretraining of the neural networks either offline or online. Illustrative embodiments perform the offline pretraining by training each respective neural network on a separate set of data that has been labeled with the corresponding user emotional state. Illustrative embodiments perform the online pretraining by training each respective neural network on data that is being streamed in real time from the virtual reality headset.

The inputs to the neural networks (i.e., recurrent neural network, convolutional neural network, and long short term memory neural network) are streams of sensor data from corresponding sensors of the plurality of sensors. The neural networks process the input streams of sensor data to generate a stream of output values. The manager neural network uses the output values to generate a stream of user emotional state predictions. A computer corresponding to the metaverse (e.g., virtual environment) or the virtual reality headset, itself, utilizes the user emotional state predictions to render an avatar associated with the user in the metaverse consistent with the predicted emotional state of the user to improve the user experience.

After illustrative embodiments train the individual neural networks, illustrative embodiments utilize the manager neural network to subsequently retrain the neural networks based on the cumulative output layer of each respective neural network. The manager neural network takes as input the emotional state predictions from the recurrent neural network, convolutional neural network, and long short term memory neural network. Illustrative embodiments train the manager neural network to minimize the error in the user emotional state predictions. The output of the manager neural network is a stream of retrained user emotional state predictions that is used by the computer corresponding to the metaverse or the virtual reality headset to render an avatar associated with the user in the metaverse consistent with the predicted emotional state of the user.

Thus, illustrative embodiments provide one or more technical solutions that overcome a technical problem with an inability of current solutions to render a current emotional state of a user in a metaverse for user awareness of what emotion the user is projecting to other in the metaverse. As a result, these one or more technical solutions provide a technical effect and practical application in the field of virtual environments.

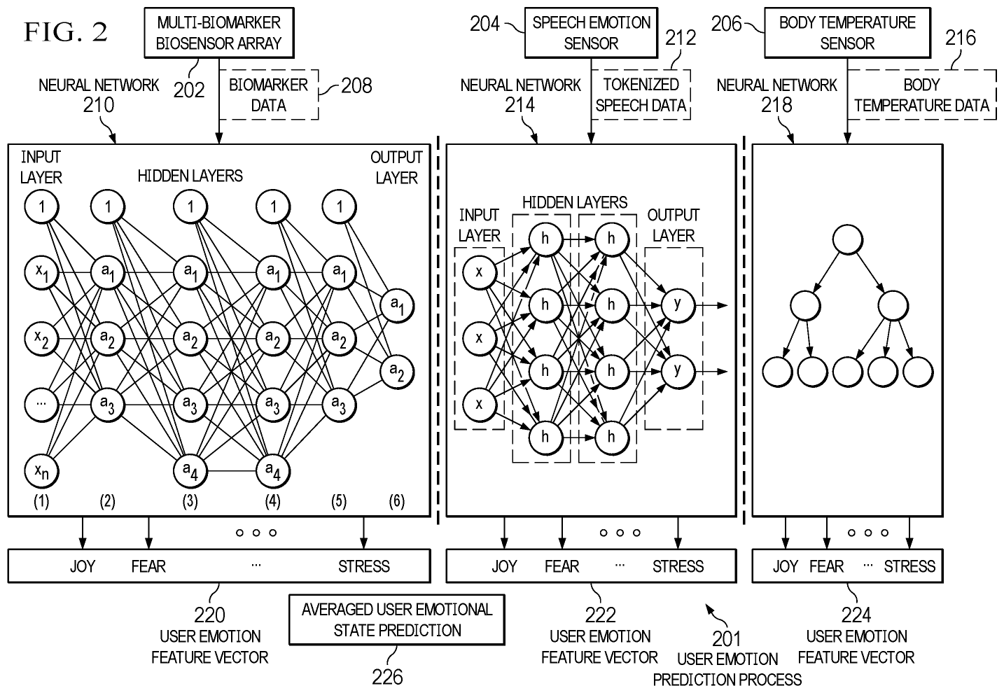

With reference now to FIG. 2, a diagram illustrating an example of a user emotion prediction process is depicted in accordance with an illustrative embodiment. User emotion prediction process 201 may be implemented in a computer, such as computer 101 in FIG. 1 or virtual reality headset 103 in FIG. 1. User emotion prediction process 201 utilizes a plurality of hardware and software components for rendering user emotion in a metaverse for user awareness.

In this example, user emotion prediction process 201 includes multi-biomarker biosensor array 202, speech emotion sensor 204, and body temperature sensor 206. Multi-biomarker biosensor array 202, speech emotion sensor 204, and body temperature sensor 206 are positioned on the virtual reality headset so as to contact the skin of a user at a set of one or more locations (e.g., at least one of forehead, base of neck, bridge of nose, and the like). Multi-biomarker biosensor array 202, speech emotion sensor 204, and body temperature sensor 206 may be, for example, sensor set 107 in FIG. 1.

Multi-biomarker biosensor array 202 outputs biomarker data 208. Biomarker data 208 contain information regarding the type and concentration of different biomarkers detected by multi-biomarker biosensor array 202 in the sweat of the user of the virtual reality device. Biomarker data 208 is input into neural network 210. Neural network 210 may be, for example, a recurrent neural network or the like. Speech emotion sensor 204 outputs tokenized speech data 212. Tokenized speech data 212 contain information regarding the type of emotion in the speech of the user. Tokenized speech data 212 is input in neural network 214. Neural network 214 may be, for example, a convolutional neural network or the like. Body temperature sensor 206 outputs body temperature data 216. Body temperature data 216 contain information regarding the body temperature of the user. Body temperature data 216 is input into neural network 218. Neural network 218 may be, for example, a long short term memory neural network or the like.

It should be noted that neural network 210, neural network 214, and neural network 218 can be located in the virtual reality headset or the computer providing the metaverse. Neural network 210 processes biomarker data 208 to output user emotion feature vector 220. Neural network 214 processes tokenized speech data 212 to output user emotion feature vector 222. Neural network 218 processes body temperature data 216 to output user emotion feature vector 224.

In this example, to increase the predictive accuracy of the user's current emotional state, the virtual reality headset or the computer providing the metaverse averages out user emotion feature vector 220, user emotion feature vector 222, and user emotion feature vector 224 to generate averaged user emotional state prediction 226. For example, illustrative embodiments can utilize a simple average of the outputs of the different neural networks. Alternatively, illustrative embodiments can utilize a weighted average of the outputs of the different neural networks.

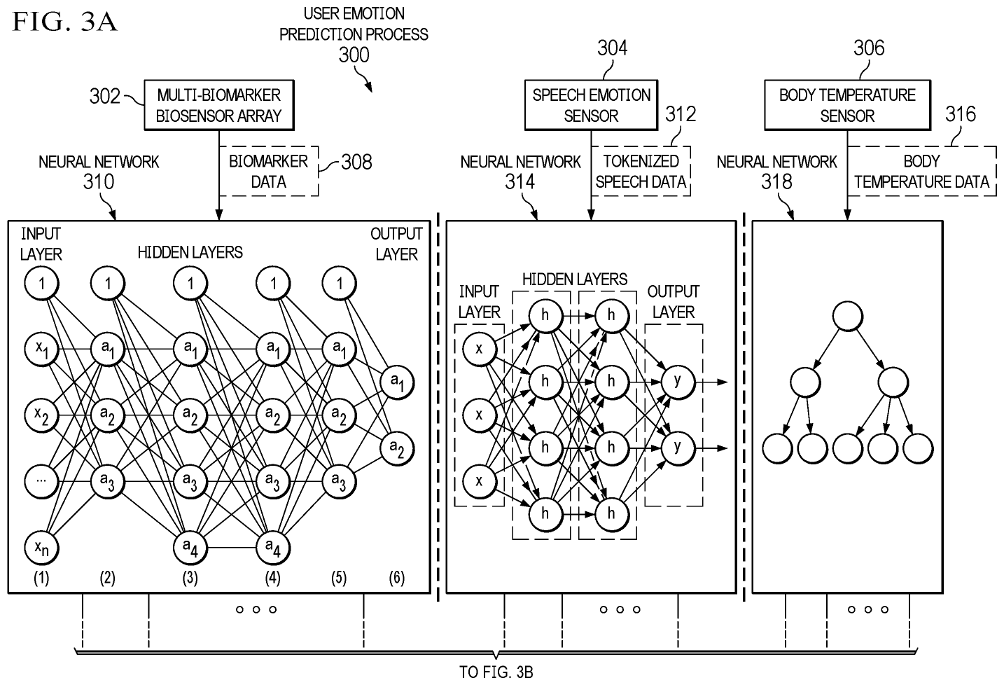

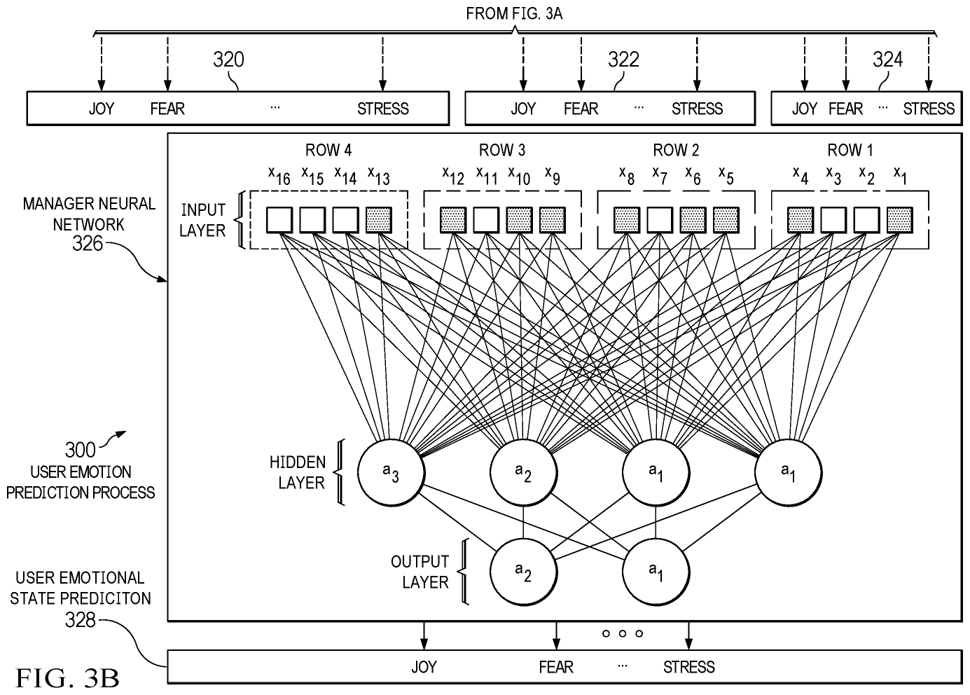

With reference now to FIGS. 3A-3B, a diagram illustrating an example of an alternative user emotion prediction process is depicted in accordance with an illustrative embodiment. User emotion prediction process 300 may be implemented in a computer, such as computer 101 in FIG. 1 or virtual reality headset 103 in FIG. 1. User emotion prediction process 300 utilizes a plurality of hardware and software components for rendering user emotion in a metaverse for user awareness.

In this example, user emotion prediction process 300 includes multi-biomarker biosensor array 302, speech emotion sensor 304, and body temperature sensor 306. Multi-biomarker biosensor array 302, speech emotion sensor 304, and body temperature sensor 306 are positioned on the virtual reality headset so as to contact the skin of a user at a set of one or more locations. Multi-biomarker biosensor array 302, speech emotion sensor 304, and body temperature sensor 306 may be, for example, multi-biomarker biosensor array 202, speech emotion sensor 204, and body temperature sensor 206 in FIG. 2.

Multi-biomarker biosensor array 302 outputs biomarker data 308. Biomarker data 308 contain information regarding the type and concentration of different biomarkers detected by multi-biomarker biosensor array 302 in the sweat of the user of the virtual reality device. Biomarker data 308 is input into neural network 310. Speech emotion sensor 304 outputs tokenized speech data 312. Tokenized speech data 312 contain information regarding the type of emotion in the speech of the user. Tokenized speech data 312 is input in neural network 314. Body temperature sensor 306 outputs body temperature data 316. Body temperature data 316 contain information regarding the body temperature of the user. Body temperature data 316 is input into neural network 318.

Neural network 310, neural network 314, and neural network 318 can be located in at least one of the virtual reality headset and the computer providing the metaverse. Neural network 310 processes biomarker data 308 to output user emotion feature vector 320. Neural network 314 processes tokenized speech data 312 to output user emotion feature vector 322. Neural network 318 processes body temperature data 316 to output user emotion feature vector 324.

In this example, to increase the predictive accuracy of the user's current emotional state, the virtual reality headset or the computer providing the metaverse inputs user emotion feature vector 320, user emotion feature vector 322, and user emotion feature vector 324 into manager neural network 326 to generate user emotional state prediction 328. Manager neural network 326 generates user emotional state prediction 328 by performing end-to-end classification of the emotional state of the user.

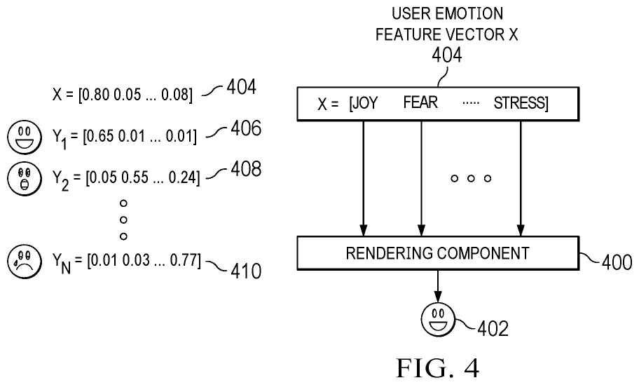

With reference now to FIG. 4, a diagram illustrating an example of a rendering component is depicted in accordance with an illustrative embodiment. Rendering component 400 may be implemented in a computer, such as computer 101 in FIG. 1 or virtual reality headset 103 in FIG. 1. Rendering component 400 can utilize a combination of hardware and software components to generate avatar 402 in a metaverse for a user of the virtual reality headset consistent with the current emotional state of the user based on user emotion feature vector X 404. User emotion feature vector X 404 can be, for example, averaged user emotional state prediction 226 in FIG. 2 or user emotional state prediction 328 in FIG. 3.

In one illustrative embodiment, rendering component 400 is a mapping function that maps user emotion feature vector X 404 corresponding to the user of the virtual reality headset to a best matching set of existing avatar vectors, such as avatar vector Y₁ 406, avatar vector Y₂ 408 . . . to avatar vector Y_{N} 410. The mapping function can be any linear mapping function or nonlinear mapping function, which includes a discriminative neural network (e.g., a supervised machine learning model). As an illustrative example, illustrative embodiments encode the set of existing avatar vectors with the same embeddings as user emotion feature vector X 404. Illustrative embodiments consider an avatar vector Y that maximizes the inner product X*Y to be the best match for user emotion feature vector X 404. In this example, illustrative embodiments determine that avatar vector Y₁ 406 is the best match for user emotion feature vector X 404 based on the values [0.65 0.01 . . . 0.01] of avatar vector Y₁ 406 being the closest matching to the values [0.80 0.05 . . . 0.08] of user emotion feature vector X 404 as opposed to the other values in the avatar vector set. It should be noted that illustrative embodiments can also utilize automated emotion feature extraction to extract emotion features from avatars based on, for example, avatar body posture, avatar facial expressions, and the like, which may be accompanied with dimensionality reduction and low-rank approximation.

In an alternative illustrative embodiment, rendering component 400 is a generative neural network that directly generates avatar 402 for the user. The generative neural network can include, for example, at least one of a decoder part of a variational auto-encoder and a generative part of a generative adversarial neural network.

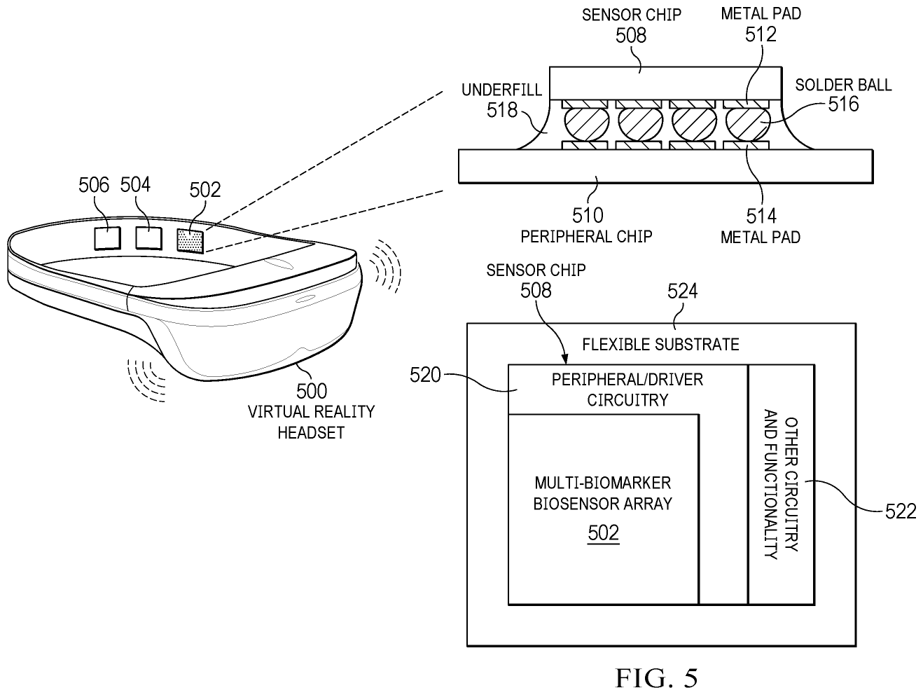

With reference now to FIG. 5, a diagram illustrating an example of a virtual reality headset is depicted in accordance with an illustrative embodiment. Virtual reality headset 500 may be, for example, virtual reality headset 103 in computing environment 100 in FIG. 1.

In this example, virtual reality headset 500 includes a plurality of sensors (i.e., multi-biomarker biosensor array 502, body temperature sensor 504, and speech emotion sensor 506). For example, multi-biomarker biosensor array 502, body temperature sensor 504, and speech emotion sensor 506 are attached to the inner portion of the virtual reality headset in positions where virtual reality headset 500 makes contact with the skin of the user (e.g., at least one of the skin of the forehead, skin at the base of the neck, skin on the bridge of the nose, and the like). Of course, alternative illustrative embodiments can utilize other skin contact points as well, in addition to or instead of, the forehead, base of the neck, and bridge of nose.

In addition, each of multi-biomarker biosensor array 502, body temperature sensor 504, and speech emotion sensor 506 can be included on its own individual sensor chip 508. Alternatively, all of multi-biomarker biosensor array 502, body temperature sensor 504, and speech emotion sensor 506 can be included on one sensor chip 508.

In one illustrative embodiment, sensor chip 508 is bonded (e.g., using flip-chip bonding such as metal pad 512, metal pad 514, solder ball 516, and underfill 518) to peripheral chip 510. Peripheral chip 510 can include further functionality, such as, for example, memory, battery, communication, processing, and the like. Moreover, peripheral/driver circuitry 520, as well as other additional circuitry and functionality 522, can be integrated monolithically on sensor chip 508. Also, sensor chip 508 (with or without a bonded chip) may be integrated heterogeneously (i.e., with separately manufactured components into a higher-level assembly, such as a system-in-package (e.g., a handheld or portable device)). In some illustrative embodiments, sensor chip 508 is bonded onto flexible substrate 524 such that sensor chip 508 can be placed on the skin of the user like a patch. For example, multi-biomarker biosensor array 502 of illustrative embodiments can be made flexible using flexible substrate 524 so that the user can wear sensor chip 508 like a patch on the inside of virtual reality headset 500.

Illustrative embodiments utilize a fabricated monolithically integrated array of a plurality of multi-biomarker biosensors. Each biomarker biosensor of the array is functionalized by treating the sensing surface of that particular biosensor for detecting one or more biomarkers in human sweat. The materials and approaches known in the art for functionalization of sensing surfaces may be used. Furthermore, multi-biomarker biosensor array 502 can include redundancy for improving fault tolerance, facilitating anomaly detection by machine learning models (e.g., neural networks), and improving signal-to-noise ratio by averaging out uncorrelated noise.

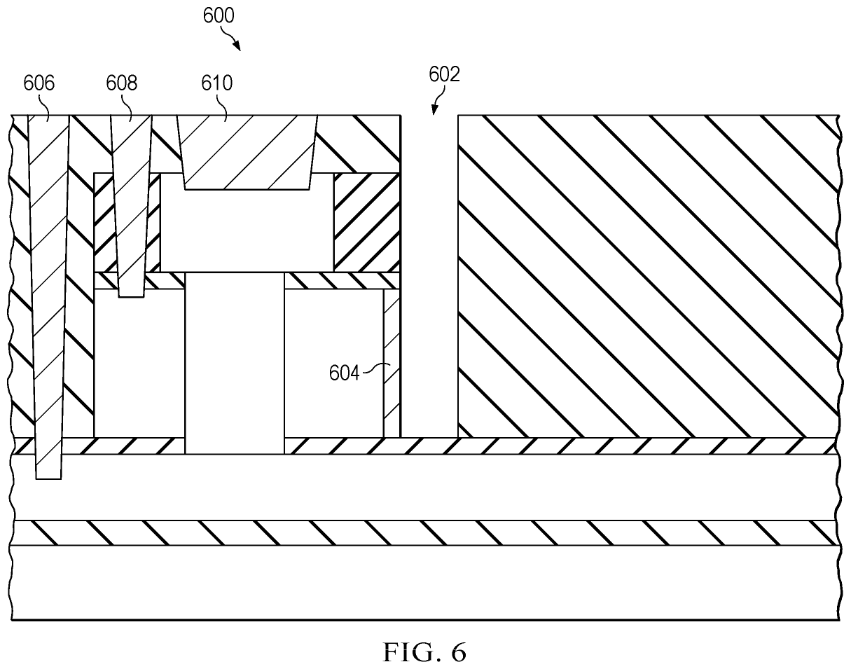

With reference now to FIG. 6, a diagram illustrating an example of a biosensor is depicted in accordance with an illustrative embodiment. Biosensor 600 is implemented in a multi-biomarker biosensor array, such as, for example, multi-biomarker biosensor array 502 attached to virtual reality headset 500 in FIG. 5. It should be noted that biosensor 600 can be one of a plurality of different biosensors implemented in the multi-biomarker biosensor array.

In this example, biosensor 600 includes sample trench 602, sensing surface 604, contact 606, contact 608, and contact 610. Sample trench 602 collects sweat from a user of the virtual reality headset. Sensing surface 604 detects the type and concentration of biomarkers contained in the sweat of the user. Contact 606, contact 608, and contact 610 are contact points.

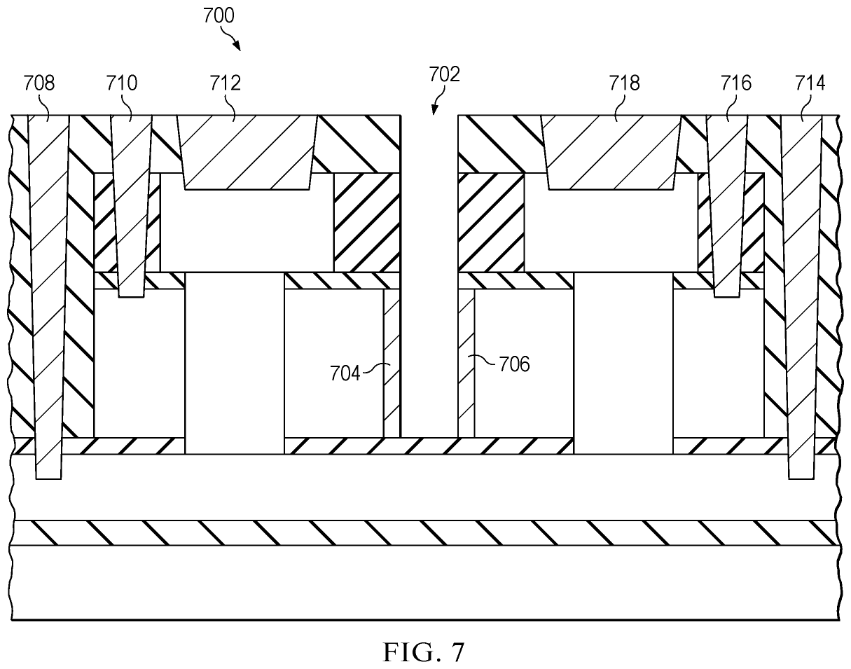

With reference now to FIG. 7, a diagram illustrating an example of an alternative biosensor is depicted in accordance with an illustrative embodiment. Biosensor 700 is implemented in a multi-biomarker biosensor array, such as, for example, multi-biomarker biosensor array 502 attached to virtual reality headset 500 in FIG. 5. It should be noted that biosensor 700 can be one of a plurality of different biosensors implemented in the multi-biomarker biosensor array.

In this example, biosensor 700 includes sample trench 702, sensing surface 704, sensing surface 706, contact 708, contact 710, contact 712, contact 714, contact 716, and contact 718. Sample trench 702 collects sweat from a user of the virtual reality headset. Sensing surface 704 and sensing surface 706 detect the type and concentration of biomarkers contained in the sweat of the user. Contact 708, contact 710, contact 712, contact 714, contact 716, and contact 718 are contact points.

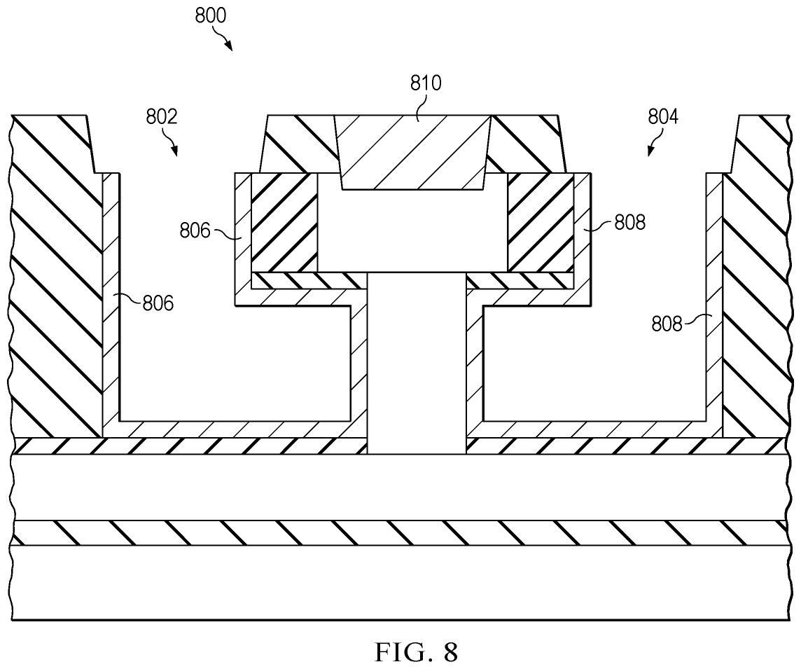

With reference now to FIG. 8, is a diagram illustrating an example of another alternative biosensor is depicted in accordance with an illustrative embodiment. Biosensor 800 is implemented in a multi-biomarker biosensor array, such as, for example, multi-biomarker biosensor array 502 attached to virtual reality headset 500 in FIG. 5. It should be noted that biosensor 800 can be one of a plurality of different biosensors implemented in the multi-biomarker biosensor array.

In this example, biosensor 800 includes sample trench 802, sample trench 804, sensing surface 806, sensing surface 808, and contact 810. Sample trench 802 and sample trench 804 collect sweat from a user of the virtual reality headset. Sensing surface 806 and sensing surface 808 detect the type and concentration of biomarkers contained in the sweat of the user. Contact 810 is a contact point.

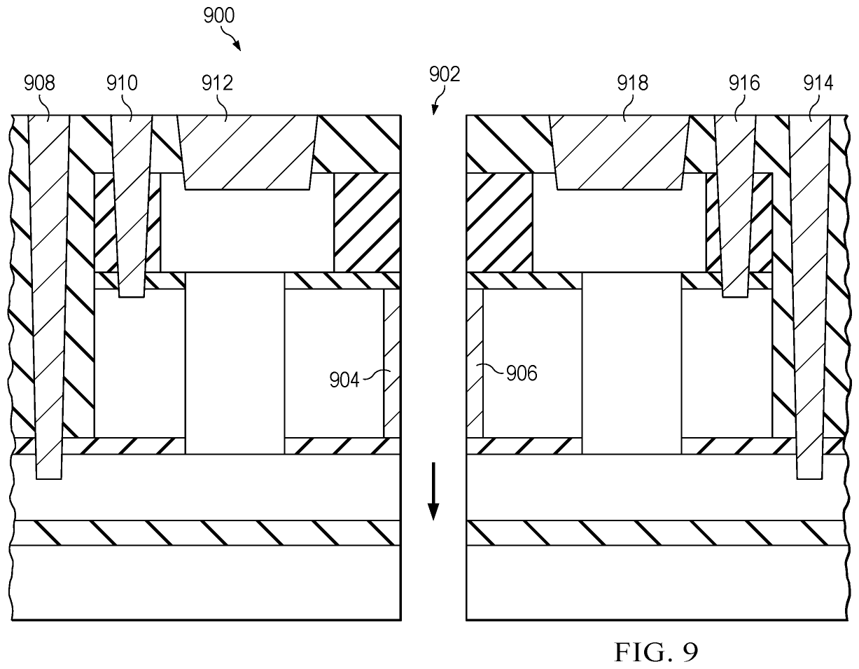

With reference now to FIG. 9, a diagram illustrating an example of another alternative biosensor is depicted in accordance with an illustrative embodiment. Biosensor 900 is implemented in a multi-biomarker biosensor array, such as, for example, multi-biomarker biosensor array 502 attached to virtual reality headset 500 in FIG. 5. It should be noted that biosensor 900 can be one of a plurality of different biosensors implemented in the multi-biomarker biosensor array.

In this example, biosensor 900 includes sample pass-through 902, sensing surface 904, sensing surface 906, contact 908, contact 910, contact 912, contact 914, contact 916, and contact 918. Sample pass-through 902 collects sweat from a user of the virtual reality headset and allows the sweat to drain out of biosensor 900. Sensing surface 904 and sensing surface 906 detect the type and concentration of biomarkers contained in the sweat of the user. Contact 908, contact 910, contact 912, contact 914, contact 916, and contact 918 are contact points.

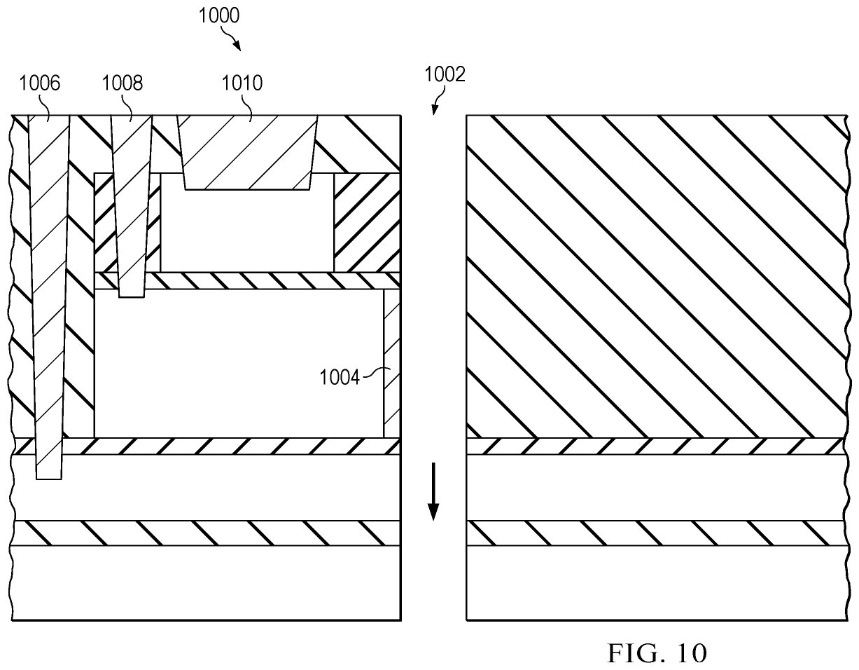

With reference now to FIG. 10, a diagram illustrating an example of another alternative biosensor is depicted in accordance with an illustrative embodiment. Biosensor 1000 is implemented in a multi-biomarker biosensor array, such as, for example, multi-biomarker biosensor array 502 attached to virtual reality headset 500 in FIG. 5. It should be noted that biosensor 1000 can be one of a plurality of different biosensors implemented in the multi-biomarker biosensor array.

In this example, biosensor 1000 includes sample pass-through 1002, sensing surface 1004, contact 1006, contact 1008, and contact 1010. Sample pass-through 1002 collects sweat from a user of the virtual reality headset and allows the sweat to drain out of biosensor 1000. Sensing surface 1004 detects the type and concentration of biomarkers contained in the sweat of the user. Contact 1006, contact 1008, and contact 1010 are contact points.

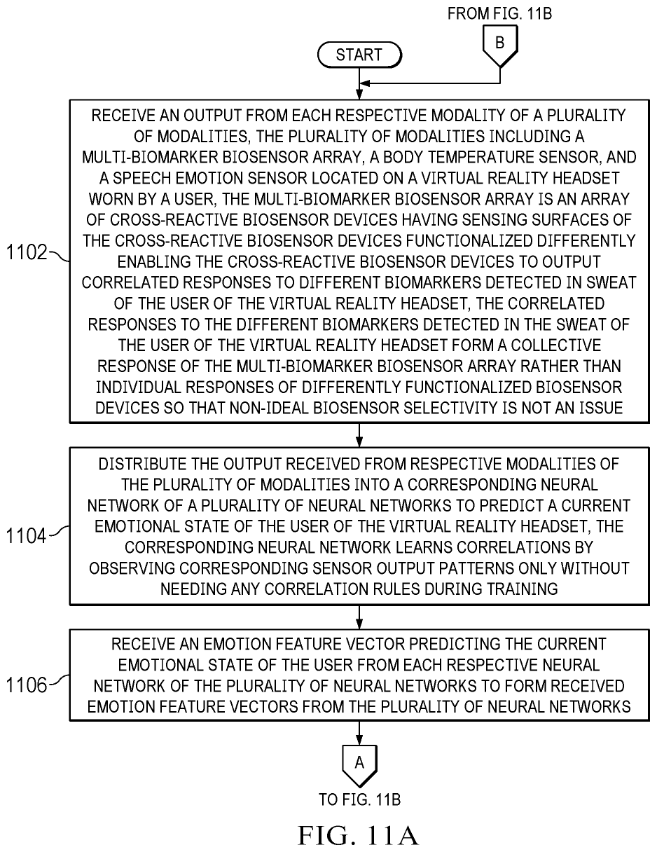

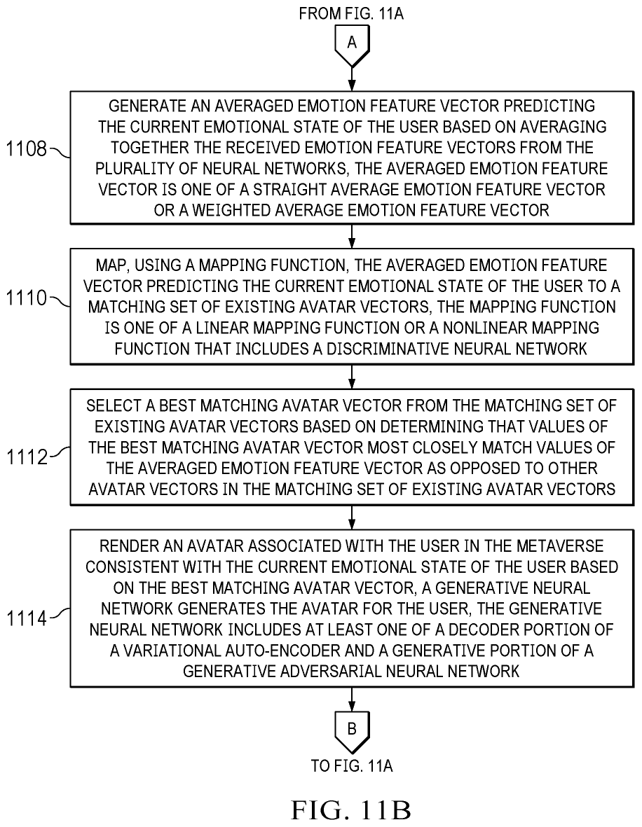

With reference now to FIGS. 11A-11B, a flowchart illustrating a process for rendering an emotional state of a virtual reality headset user in a virtual reality environment is shown in accordance with an illustrative embodiment. The process shown in FIGS. 11A-11B may be implemented in a computer, such as, for example, computer 101 in FIG. 1 or a virtual reality headset, such as, for example, virtual reality headset 103 in FIG. 1 or virtual reality headset 500 in FIG. 5. For example, the process shown in FIGS. 11A-11B may be implemented in user emotion rendering code 200 in FIG. 1.

The process begins when the computer or the virtual reality headset receives an output from each respective modality of a plurality of modalities (step 1102). The plurality of modalities includes a multi-biomarker biosensor array, a body temperature sensor, and a speech emotion sensor located on the virtual reality headset worn by a user. The multi-biomarker biosensor array is an array of cross-reactive biosensor devices having sensing surfaces of the cross-reactive biosensor devices functionalized differently, which enables the cross-reactive biosensor devices to output correlated responses to different biomarkers detected in sweat of the user of the virtual reality headset. The correlated responses to the different biomarkers detected in the sweat of the user of the virtual reality headset form a collective response of the multi-biomarker biosensor array rather than individual responses of differently functionalized biosensor devices so that non-ideal biosensor selectivity is not an issue.

The computer or the virtual reality headset distributes the output received from respective modalities of the plurality of modalities into a corresponding neural network of a plurality of neural networks to predict a current emotional state of the user of the virtual reality headset (step 1104). The corresponding neural network learns correlations by observing corresponding sensor output patterns only without needing any correlation rules during training. The computer or the virtual reality headset receives an emotion feature vector predicting the current emotional state of the user from each respective neural network of the plurality of neural networks to form received emotion feature vectors from the plurality of neural networks (step 1106).

The computer or the virtual reality headset generates an averaged emotion feature vector predicting the current emotional state of the user based on averaging together the received emotion feature vectors from the plurality of neural networks (step 1108). The averaged emotion feature vector is one of a straight average emotion feature vector or a weighted average emotion feature vector. The computer or the virtual reality headset, using a mapping function, maps the averaged emotion feature vector predicting the current emotional state of the user to a matching set of existing avatar vectors (step 1110). The mapping function is one of a linear mapping function or a nonlinear mapping function that includes a discriminative neural network.

The computer or the virtual reality headset selects a best matching avatar vector from the matching set of existing avatar vectors based on determining that values of the best matching avatar vector most closely match values of the averaged emotion feature vector as opposed to other avatar vectors in the matching set of existing avatar vectors (step 1112). The computer or the virtual reality headset renders an avatar associated with the user in the metaverse consistent with the current emotional state of the user based on the best matching avatar vector (step 1114). The computer or the virtual reality headset utilizes a generative neural network to generate the avatar for the user. The generative neural network includes at least one of a decoder portion of a variational auto-encoder and a generative portion of a generative adversarial neural network. Thereafter, the process returns to step 1102 where the computer or virtual reality headset continues to receive output from each respective modality of the plurality of modalities.

Thus, illustrative embodiments of the present disclosure provide a computer-implemented method, computer system, and computer program product for rendering user emotion in a metaverse for user awareness. The descriptions of the various embodiments of the present disclosure have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A computer-implemented method for rendering an emotional state of a virtual reality headset user, the computer-implemented method comprising:
mapping, using a mapping function, an emotion feature vector predicting a current emotional state of a user of a virtual reality headset to a matching set of existing avatar vectors;
selecting a best matching avatar vector from the matching set of existing avatar vectors based on determining that values of the best matching avatar vector most closely match values of the emotion feature vector predicting the current emotional state of the user of the virtual reality headset; and
rendering an avatar associated with the user in a metaverse consistent with the current emotional state of the user of the virtual reality headset based on the best matching avatar vector to the emotion feature vector predicting the current emotional state of the user.

2. The computer-implemented method of claim 1, further comprising:
utilizing a generative neural network to generate the avatar for the user.

3. The computer-implemented method of claim 2, wherein the generative neural network includes at least one of a decoder portion of a variational auto-encoder and a generative portion of a generative adversarial neural network.

4. The computer-implemented method of claim 1, further comprising:
receiving an output from each respective modality of a plurality of modalities, the plurality of modalities includes a multi-biomarker biosensor array, a body temperature sensor, and a speech emotion sensor located on the virtual reality headset worn by the user, the multi-biomarker biosensor array is an array of cross-reactive biosensor devices having sensing surfaces of the cross-reactive biosensor devices functionalized differently enabling the cross-reactive biosensor devices to output correlated responses to different biomarkers detected in sweat of the user of the virtual reality headset.

5. The computer-implemented method of claim 4, wherein the correlated responses to the different biomarkers detected in the sweat of the user of the virtual reality headset form a collective response of the multi-biomarker biosensor array rather than individual responses of differently functionalized biosensor devices so that non-ideal biosensor selectivity is not an issue.

6. The computer-implemented method of claim 4, further comprising:
distributing the output received from respective modalities of the plurality of modalities into a corresponding neural network of a plurality of neural networks to predict the current emotional state of the user of the virtual reality headset, the plurality of neural networks includes a recurrent neural network, a convolutional neural network, and a long short term memory neural network, the recurrent neural network processes output of the multi-biomarker biosensor array, the convolutional neural network processes output of the speech emotion sensor, and the long short term memory neural network processes output of the body temperature sensor.

7. The computer-implemented method of claim 6, wherein the corresponding neural network learns correlations by observing corresponding sensor output patterns only, without needing a correlation rule during training.

8. The computer-implemented method of claim 1, further comprising:
receiving the emotion feature vector predicting the current emotional state of the user from each respective neural network of a plurality of neural networks to form received emotion feature vectors from the plurality of neural networks; and
generating an averaged emotion feature vector predicting the current emotional state of the user based on averaging together received emotion feature vectors from the plurality of neural networks, the averaged emotion feature vector is one of a straight average emotion feature vector or a weighted average emotion feature vector.

9. The computer-implemented method of claim 1, wherein the mapping function is one of a linear mapping function or a nonlinear mapping function that includes a discriminative neural network.

10. The computer-implemented method of claim 1, wherein one of the virtual reality headset or a computer that provides the metaverse performs the computer-implemented method.

11. A computing environment for rendering an emotional state of a virtual reality headset user, the computing environment comprising:
a communication fabric;
a set of storage devices connected to the communication fabric, wherein the set of storage devices stores program instructions; and
a set of processors connected to the communication fabric, wherein the set of processors executes the program instructions to:
map, using a mapping function, an emotion feature vector predicting a current emotional state of a user of a virtual reality headset to a matching set of existing avatar vectors;
select a best matching avatar vector from the matching set of existing avatar vectors based on determining that values of the best matching avatar vector most closely match values of the emotion feature vector predicting the current emotional state of the user of the virtual reality headset; and
render an avatar associated with the user in a metaverse consistent with the current emotional state of the user of the virtual reality headset based on the best matching avatar vector to the emotion feature vector predicting the current emotional state of the user.

12. The computing environment of claim 11, wherein the set of processors further executes the program instructions to:
utilize a generative neural network to generate the avatar for the user.

13. The computing environment of claim 12, wherein the generative neural network includes at least one of a decoder portion of a variational auto-encoder and a generative portion of a generative adversarial neural network.

14. The computing environment of claim 11, wherein the set of processors further executes the program instructions to:
receive an output from each respective modality of a plurality of modalities, the plurality of modalities includes a multi-biomarker biosensor array, a body temperature sensor, and a speech emotion sensor located on the virtual reality headset worn by the user, the multi-biomarker biosensor array is an array of cross-reactive biosensor devices having sensing surfaces of the cross-reactive biosensor devices functionalized differently enabling the cross-reactive biosensor devices to output correlated responses to different biomarkers detected in sweat of the user of the virtual reality headset.

15. A computer program product for rendering an emotional state of a virtual reality headset user, the computer program product comprising a computer-readable storage medium having program instructions embodied therewith, the program instructions executable by a set of processors to cause the set of processors to:
map, using a mapping function, an emotion feature vector predicting a current emotional state of a user of a virtual reality headset to a matching set of existing avatar vectors;
select a best matching avatar vector from the matching set of existing avatar vectors based on determining that values of the best matching avatar vector most closely match values of the emotion feature vector predicting the current emotional state of the user of the virtual reality headset; and
render an avatar associated with the user in a metaverse consistent with the current emotional state of the user of the virtual reality headset based on the best matching avatar vector to the emotion feature vector predicting the current emotional state of the user.

16. The computer program product of claim 15, wherein the program instructions further cause the set of processors to:
utilize a generative neural network to generate the avatar for the user.

17. The computer program product of claim 16, wherein the generative neural network includes at least one of a decoder portion of a variational auto-encoder and a generative portion of a generative adversarial neural network.

18. The computer program product of claim 15, wherein the program instructions further cause the set of processors to:
receive an output from each respective modality of a plurality of modalities, the plurality of modalities includes a multi-biomarker biosensor array, a body temperature sensor, and a speech emotion sensor located on the virtual reality headset worn by the user, the multi-biomarker biosensor array is an array of cross-reactive biosensor devices having sensing surfaces of the cross-reactive biosensor devices functionalized differently enabling the cross-reactive biosensor devices to output correlated responses to different biomarkers detected in sweat of the user of the virtual reality headset.

19. The computer program product of claim 18, wherein the correlated responses to the different biomarkers detected in the sweat of the user of the virtual reality headset form a collective response of the multi-biomarker biosensor array rather than individual responses of differently functionalized biosensor devices so that non-ideal biosensor selectivity is not an issue.

20. The computer program product of claim 18, wherein the program instructions further cause the set of processors to:
distribute the output received from respective modalities of the plurality of modalities into a corresponding neural network of a plurality of neural networks to predict the current emotional state of the user of the virtual reality headset, the plurality of neural networks includes a recurrent neural network, a convolutional neural network, and a long short term memory neural network, the recurrent neural network processes output of the multi-biomarker biosensor array, the convolutional neural network processes output of the speech emotion sensor, and the long short term memory neural network processes output of the body temperature sensor.
